SEQUENCE LISTING (8 sequences)

SEQ ID NO: 1 - protein, 412 aa, Danio rerio
MAEMYIRVAEEENEEPMEIPSEDDGTVLLSTVSAQFPGACGLRFRSPVSQCMRGVRLVDGILHAPENGWGNLVYVVNYPKETVLPDNKRKMDEIDASSATKIKRGDQKTSDLIVLGLPWKTSEQDLKDYFGTFGEVIMVQVKRDVKTGNSKGFGFVRFGDWETQSKVMTQRHMIDGRWCDCKLPNSKQGIDEPMRSRKVFVGRCTEDMTADELRQFFMQYGEVTDVFIPKPFRAFAFVTFADDQVAAALCGEDLIIKGVSVHISNAEPKHNNTRQMMERAGRFGNGFGGQGFAGSRSNMGGGGGGSSSSLGNFGNFNLNPAMMAAAQAALQSSWGMMGMLAQQNQSGTSGTSTSGTSSSRDQAQTYSSANSNYGSSSAALGWGTGSNSGAASAGFNSSFGSSMESKSSGWGM

SEQ ID NO: 2 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 3 - protein, 401 aa, Danio rerio
MTECYIRVAEDENEEPMEIPSEEDGTVLLSTVAAQFPGACGLRYRSPVSQCMRGVRLVEGVLHAPEADWGNLVYVVNYPKDNKRKMDEMDAASAVKIKRGIQKTSDLIVLGLPWKTTEQDLKDYFGTFGEVIMVQVKRDAKSGNSKGFGFVRFTDYETQIKVMSQRHMIDGRWCDCKLPNSKYFLEQAGPDEPMRSRKVFVGRCTEDMTADELRQFFMQYGEVTDVFIPKPFRAFAFVTFADDQVAQSLCGEDLIIKGTSVHISNAEPKHNNSRQMMDRGRFGGYGGQGFGSSRSPNSNVNFGALNLNPAMMAAAQAALQSSWGMMGMLANQQNQTPASGTNPSGQSGSRDQTQNYSASSNNYNAGSSAALGWGAGTNSAAAGGFNSSFGSSMETKSSWGM

SEQ ID NO: 4 - protein, 498 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 5 - protein, 1142 aa, Artificial Sequence
  Fusion Protein
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGAMAEMYIRVAEEENEEPMEIPSEDDGTVLLSTVSAQFPGACGLRFRSPVSQCMRGVRLVDGILHAPENGWGNLVYVVNYPKETVLPDNKRKMDEIDASSATKIKRGDQKTSDLIVLGLPWKTSEQDLKDYFGTFGEVIMVQVKRDVKTGNSKGFGFVRFGDWETQSKVMTQRHMIDGRWCDCKLPNSKQGIDEPMRSRKVFVGRCTEDMTADELRQFFMQYGEVTDVFIPKPFRAFAFVTFADDQVAAALCGEDLIIKGVSVHISNAEPKHNNTRQMMERAGRFGNGFGGQGFAGSRSNMGGGGGGSSSSLGNFGNFNLNPAMMAAAQAALQSSWGMMGMLAQQNQSGTSGTSTSGTSSSRDQAQTYSSANSNYGSSSAALGWGTGSNSGAASAGFNSSFGSSMESKSSGWGMTRDISIEMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 6 - DNA, 1239 bp, Danio rerio
atggccgagatgtacattcgagttgcggaggaggagaatgaggagccaatggagatcccgtcagaggatgatggcacggttttgctttctacagtgtccgctcagtttccaggggcttgtggtctgcgcttccggagcccagtgtctcagtgcatgaggggggttcgtctggtggacggcattctgcatgcacctgagaacggctggggaaacctagtctatgtggttaattacccaaaagaaactgttctgccagataataagaggaagatggatgagatcgatgcttcatctgcgaccaagatcaagagaggagatcagaagacttcagatctgattgtgctgggtctgccatggaagacttcagagcaagacttaaaagactacttcggtacatttggggaagtcatcatggtgcaggtcaagcgggatgtgaagacaggaaattcaaaagggtttggctttgtgaggtttggagactgggagactcagagtaaggtgatgacacagcggcacatgattgatggccggtggtgcgactgtaaactgcccaactcaaagcaaggtatagatgaaccaatgaggagccgtaaagtgtttgtgggccgctgcacagaggacatgactgccgatgagctccgtcagttcttcatgcagtacggagaggtcacagacgtctttattcccaaaccgttcagagcgtttgcttttgtcacctttgcagatgaccaggttgccgccgctttgtgtggagaagatctgatcatcaagggcgtcagtgtgcatatctcaaacgctgagcccaaacacaataacactaggcagatgatggagcgggcagggcgctttgggaatgggttcggaggtcagggttttgcaggcagccgaagcaacatgggtggtggtggtgggggtagctccagcagcttgggaaattttggcaatttcaatctaaacccggccatgatggctgccgcccaggctgccttgcagagtagttggggtatgatgggaatgctagctcagcagaatcagtcgggtacttcaggcacaagcacaagtggcaccagttcctctcgagaccaagcccaaacatatagctcggctaacagcaattacggcagcagctcagctgctctcggctggggcaccggctctaactcgggcgctgccagtgctggctttaactccagttttggctctagtatggagtccaagtcatcggggtggggtatgtaa

SEQ ID NO: 7 - DNA, 1494 bp, Arabidopsis thaliana
atgaagatggacaagaagaccatcgtgtggtttagaagagacctgagaatcgaggataaccctgctctggccgccgctgcccacgaaggatctgtgttccctgtgttcatctggtgtccagaggaggagggacagttctaccctggaagagcctctagatggtggatgaagcagagcctggcccacctgagccagtctctgaaagctctgggatctgacctgacactgatcaaaacccacaacaccatctctgctatcctggattgcatcagagtgacaggagctaccaaagtggtgttcaaccacctgtacgatccagtgtctctggtgagagaccacaccgtgaaagagaaactggtggagagaggcatcagcgtgcagagctacaacggagacctgctgtacgagccatgggaaatctactgcgagaagggcaagccattcacaagcttcaactcttactggaaaaagtgcctggacatgagcatcgagagcgtgatgctgcctcctccatggagactgatgcctatcacagctgccgccgaggctatctgggcttgttctatcgaggagctgggcctggagaacgaggctgagaaaccatctaacgctctgctgacaagagcctggagccctggctggtctaacgctgataaactgctgaacgagttcatcgagaagcagctgatcgattacgctaaaaacagcaagaaggtggtgggaaactctacatctctgctgtctccatacctgcactttggcgagatcagcgtgagacacgtgttccagtgtgctagaatgaaacagatcatctgggccagagacaaaaacagcgagggagaggagagcgctgatctgtttctgagaggcatcggactgagagagtacagcagatacatctgtttcaacttcccattcacccacgagcagagcctgctgagccacctgagattctttccatgggatgccgacgtggacaaattcaaagcttggagacagggcagaacaggataccctctggtggacgccggaatgagagagctgtgggccaccggctggatgcacaacagaatcagagtgatcgtgagctctttcgctgtgaagtttctgctgctgccttggaagtggggaatgaaatacttctgggacaccctgctggacgctgacctggagtgcgacatcctgggatggcagtacatcagcggatctatcccagacggacacgagctggacagactggacaacccagctctgcagggagctaaatacgatcctgagggcgagtacatcagacagtggctgccagagctggctagactgccaaccgagtggatccaccacccttgggatgctccactgacagtgctgaaggctagcggcgtggagctgggcaccaactacgccaaacctatcgtggacatcgacaccgctagagagctgctggccaaagccatcagcagaaccagaggagcccagatcatgatcggagctgct

SEQ ID NO: 8 - DNA, 3426 bp, Artificial Sequence
  optoTDP-43 DNA
atggctagcagcgaggatgtgatcaaagagttcatgagattcaaagtgagaatggaaggcagcgtgaacggacacgagttcgagatcgagggagaaggcgaaggaagaccttacgagggaactcagacagctaaactgaaggtcacaaagggaggacctctgcctttcgcttgggacatcctgagccctcagttccagtacggaagcaaggcttacgtgaaacaccctgctgacatccctgactacctgaaactgagcttcccagaaggattcaagtgggagagagtgatgaactttgaggacggtggagtggtgactgtgacacaggatagcagcctgcaagacggagaattcatctacaaggtcaaactgagaggaaccaattttcccagcgatggtcctgtgatgcagaaaaagacaatgggttgggaggccagcacagagagaatgtatccagaggatggagccctgaagggcgagatcaaaatgagactgaagctgaaagatggcggacactacgacgctgaggtgaagacaacatacatggctaagaagcctgtgcaactgcctggcgcttacaagacagatatcaagctggacatcactagccacaacgaagactataccatcgtcgagcagtacgaaagagccgaaggtagacatagcacaggtgcaatggccgagatgtacattcgagttgcggaggaggagaatgaggagccaatggagatcccgtcagaggatgatggcacggttttgctttctacagtgtccgctcagtttccaggggcttgtggtctgcgcttccggagcccagtgtctcagtgcatgaggggggttcgtctggtggacggcattctgcatgcacctgagaacggctggggaaacctagtctatgtggttaattacccaaaagaaactgttctgccagataataagaggaagatggatgagatcgatgcttcatctgcgaccaagatcaagagaggagatcagaagacttcagatctgattgtgctgggtctgccatggaagacttcagagcaagacttaaaagactacttcggtacatttggggaagtcatcatggtgcaggtcaagcgggatgtgaagacaggaaattcaaaagggtttggctttgtgaggtttggagactgggagactcagagtaaggtgatgacacagcggcacatgattgatggccggtggtgcgactgtaaactgcccaactcaaagcaaggtatagatgaaccaatgaggagccgtaaagtgtttgtgggccgctgcacagaggacatgactgccgatgagctccgtcagttcttcatgcagtacggagaggtcacagacgtctttattcccaaaccgttcagagcgtttgcttttgtcacctttgcagatgaccaggttgccgccgctttgtgtggagaagatctgatcatcaagggcgtcagtgtgcatatctcaaacgctgagcccaaacacaataacactaggcagatgatggagcgggcagggcgctttgggaatgggttcggaggtcagggttttgcaggcagccgaagcaacatgggtggtggtggtgggggtagctccagcagcttgggaaattttggcaatttcaatctaaacccggccatgatggctgccgcccaggctgccttgcagagtagttggggtatgatgggaatgctagctcagcagaatcagtcgggtacttcaggcacaagcacaagtggcaccagttcctctcgagaccaagcccaaacatatagctcggctaacagcaattacggcagcagctcagctgctctcggctggggcaccggctctaactcgggcgctgccagtgctggctttaactccagttttggctctagtatggagtccaagtcatcggggtggggtatgacgcgtgatatctcgatcgagatgaagatggacaagaagaccatcgtgtggtttagaagagacctgagaatcgaggataaccctgctctggccgccgctgcccacgaaggatctgtgttccctgtgttcatctggtgtccagaggaggagggacagttctaccctggaagagcctctagatggtggatgaagcagagcctggcccacctgagccagtctctgaaagctctgggatctgacctgacactgatcaaaacccacaacaccatctctgctatcctggattgcatcagagtgacaggagctaccaaagtggtgttcaaccacctgtacgatccagtgtctctggtgagagaccacaccgtgaaagagaaactggtggagagaggcatcagcgtgcagagctacaacggagacctgctgtacgagccatgggaaatctactgcgagaagggcaagccattcacaagcttcaactcttactggaaaaagtgcctggacatgagcatcgagagcgtgatgctgcctcctccatggagactgatgcctatcacagctgccgccgaggctatctgggcttgttctatcgaggagctgggcctggagaacgaggctgagaaaccatctaacgctctgctgacaagagcctggagccctggctggtctaacgctgataaactgctgaacgagttcatcgagaagcagctgatcgattacgctaaaaacagcaagaaggtggtgggaaactctacatctctgctgtctccatacctgcactttggcgagatcagcgtgagacacgtgttccagtgtgctagaatgaaacagatcatctgggccagagacaaaaacagcgagggagaggagagcgctgatctgtttctgagaggcatcggactgagagagtacagcagatacatctgtttcaacttcccattcacccacgagcagagcctgctgagccacctgagattctttccatgggatgccgacgtggacaaattcaaagcttggagacagggcagaacaggataccctctggtggacgccggaatgagagagctgtgggccaccggctggatgcacaacagaatcagagtgatcgtgagctctttcgctgtgaagtttctgctgctgccttggaagtggggaatgaaatacttctgggacaccctgctggacgctgacctggagtgcgacatcctgggatggcagtacatcagcggatctatcccagacggacacgagctggacagactggacaacccagctctgcagggagctaaatacgatcctgagggcgagtacatcagacagtggctgccagagctggctagactgccaaccgagtggatccaccacccttgggatgctccactgacagtgctgaaggctagcggcgtggagctgggcaccaactacgccaaacctatcgtggacatcgacaccgctagagagctgctggccaaagccatcagcagaaccagaggagcccagatcatgatcggagctgct

(19) United States
(12) Patent Application Publication
(10) Pub. No.: US 2021/0363202 A1
(43) Pub. Date: Nov. 25, 2021

(54) FUSION PROTEIN AND UTILIZATION THEREOF

(71) Applicant: Inter-University Research Institute Corporation Research Organization of Information and Systems, Tokyo (JP)

(72) Inventors: Kazuhide ASAKAWA, Mishima-shi (JP); Koicbi KAWAKAMI, Mishima-shi (JP)

(21) Appl. No.: 17/281,550

(22) PCT Filed: Sep. 26, 2019
(86) PCT No.: PCT/JP2019/037829

§ 371 Date: Mar. 30, 2021

(30) Foreign Application Priority Data
Oct. 1, 2018 (JP) 2018-186569

(51) Int. Cl. C07K 14/46; A01K 67/027; G01N 21/76; G01N 21/01

(52) U.S. Cl. CPC C07K 14/461; A01K 67/0275; G01N 21/763; C07K 14/415; A01K 2267/0356; A01K 2227/40; G01N 21/01

(57) ABSTRACT

What is provided is a fusion protein including any one of proteins of (A) to (C) below; and a tag protein that dimerizes or multimerizes in response to a stimulus, (A) a protein that consists of an amino acid sequence represented by SEQ ID NO: 1;
(B) a protein that consists of an amino acid sequence having 70% or more identity with the amino acid sequence represented by SEQ ID NO: 1 and has an aggregate-forming ability, and
(C) a protein that consists of an amino acid sequence obtained by performing deletion, substitution, insertion, or addition of one or several amino acids with respect to the amino acid sequence represented by SEQ ID NO: 1 and has the aggregate-forming ability.

### TECHNICAL FIELD

The present invention relates to a fusion protein and utilization thereof.

Priority is claimed on Japanese Patent Application No. 2018-186569, filed on Oct. 1, 2018, the content of which is incorporated herein by reference.

### BACKGROUND ART

In neurodegenerative diseases such as amyotrophic lateral sclerosis (hereinafter also referred to as ALS) and frontotemporal lobar degeneration (hereinafter also referred to as FTLD), it is known that inclusion bodies containing aggregated RNA-binding protein TDP-43 (TAR DNA-binding protein of 43 kDa) as a main component accumulate in the cytoplasm of degenerating nerve cells in the brain and spinal cord. Disease groups such as ALS and FTLD are collectively referred to as TDP-43 proteinopathies.

It is expected that TDP-43 is closely involved in the onset and progression of the TDP-43 proteinopathies, mainly based on the accumulation of the inclusion bodies containing TDP-43 as a main component. It is expected that a cure for TDP-43 proteinopathies can be developed from viewpoints of functional modulation of the TDP-43, inhibition of TDP-43 aggregation, and removal of TDP-43 aggregates.

As described above, pathological features of TDP-43 proteinopathies include the accumulation of TDP-43 aggregates, and disease models reflecting such a feature have been proposed (see, for example, Patent Document 1).

### CITATION LIST

### Patent Literature

### Patent Document 1

Japanese Unexamined Patent Application, First Publication No. 2014-171425

### SUMMARY OF INVENTION

### Technical Problem

However, in the most ALS cases, genetic mutations in the TDP-43 gene or overexpression of the TDP-43 protein has not been observed. Therefore, for example, as in a disease model described in Patent Document 1, there was a concern that a phenotype of the disease model manifested by an introduction of amino acid substitution mutation into TDP-43 and the overexpression of the resulting mutant TDP-43 did not necessarily reflect pathological conditions.

The present invention has been made in view of the above circumstances, and provides a TDP-43 proteinopathy model that reflects pathological conditions of proteinopathies, a fusion protein, a gene, a vector, a cell, and a non-human animal for creating the model, and a screening method, a screening kit, and a screening apparatus using the model.

### Solution to Problem

As a result of examining the multimerization status and intracellular localization of TDP-43 protein, the present inventors created a modified TDP-43 protein that behaves more closely to pathophysiological conditions in vivo, and completed the present invention.

That is, the present invention includes the following aspects.

[1] A fusion protein including:

any one of proteins of (A) to (C) below; and

a tag protein that dimerizes or multimerizes in response to a stimulus,

(A) a protein that consists of an amino acid sequence represented by SEQ ID NO: 1,

(B) a protein that consists of an amino acid sequence having 70% or more identity with the amino acid sequence represented by SEQ ID NO: 1 and has an aggregate-forming ability, and

(C) a protein that consists of an amino acid sequence obtained by performing deletion, substitution, insertion, or addition of one or several amino acids with respect to the amino acid sequence represented by SEQ ID NO: 1 and has the aggregate-forming ability.

[2] The fusion protein according to [1], further including a labeled protein.

[3] A gene encoding the fusion protein according to [1] or [2].

[4] A vector including the gene according to [3].

[5] A cell including: the fusion protein according to [1] or [2], the gene according to [3] or a transcript thereof, or the vector according to [4].

[6] The cell according to [5], in which expressions of a tardbp gene or a homolog thereof, and a tardbpl gene or a homolog thereof are suppressed or lost, or functions of a Tardbp protein or a homolog thereof and a Tardbpl protein or a homolog thereof are suppressed or lost.

[7] A non-human animal including: the fusion protein according to [1] or [2], the gene according to [3] or a transcript thereof, or the vector according to [4].

[8] The non-human animal according to [7], in which expressions of a tardbp gene or a homolog thereof, and a tardbpl gene or a homolog thereof are suppressed or lost, or functions of a Tardbp protein or a homolog thereof and a Tardbpl protein or a homolog thereof are suppressed or lost.

[9] A TDP-43 proteinopathy model, which is the cell according to [5] or [6], or the non-human animal according to [7] or [8], in which the protein forms a dimer or a multimer in response to a stimulus.

[10] The TDP-43 proteinopathy model according to [9], which is a model of amyotrophic lateral sclerosis or frontotemporal lobar degeneration.

[11] A screening method including:

bringing or administrating the cell according to [5] or [6] or the non-human animal according to [7] or [8] into contact with or with a test substance under the presence of a stimulus; and

selecting a useful candidate substance for preventing or treating TDP-43 proteinopathies.

[12] A prophylactic drug or therapeutic drug screening kit for TDP-43 proteinopathies, the kit including:

the cell according to [5] or [6] or the non-human animal according to [7] or [8].

[13] A prophylactic drug or therapeutic drug screening apparatus for proteinopathies, the apparatus including: the cell according to [5] or [6] or the non-human animal according to [7] or [8]; a well plate containing any of the cell or the non-human animal; and a light illumination device.

### Advantageous Effects of Invention

According to the present invention, it is possible to provide a proteinopathy model that reflects pathological conditions of TDP-43 proteinopathies.

### BRIEF DESCRIPTION OF DRAWINGS

FIG. 1 is a schematic configuration diagram showing an example of a screening apparatus of the present embodiment.

FIG. 2 is a schematic configuration diagram showing an example of a fusion protein of the present invention.

FIG. 3 is a photograph of a wild-type zebrafish and a zebrafish (tardbp −/−, tardbpl −/−) in which a tardbp gene and a tardbpl gene are knocked out.

FIG. 4 is a graph showing a recovery of blood circulation in a heart of the zebrafish (tardbp −/−, tardbpl −/−) by optoTDP-43.

FIG. 5 shows observation results of a transfer of a TDP-43 protein to a cytoplasm by blue light illumination.

FIG. 6 shows observation results of the transfer of TDP-43 protein to the cytoplasm by blue light illumination, in a zebrafish in which a fusion protein is expressed specifically for spinal cord neurons.

FIG. 7 shows observation results of an axon elongation by blue light illumination, in a zebrafish in which a fusion protein is expressed specifically for spinal cord neurons.

FIG. 8A shows an observation result of axon side branches of a zebrafish in which optoTDP-43 is expressed only in spinal motor neurons.

FIG. 8B is a graph showing the number of branches of the axon side branches of the zebrafish in which optoTDP-43 is expressed only in spinal motor neurons.

FIG. 8C shows observation results showing localization of presynapses and postsynapses, in the zebrafish in which optoTDP-43 is expressed only in spinal motor neurons.

FIG. 9A shows observation results showing an induction of aggregation of optoTDP-43 in the cytoplasm by blue light illumination, in a zebrafish expressing the optoTDP-43 in almost all motor neurons.

FIG. 9B shows observation results showing an induction of aggregation of optoTDP-43 and endogenous TDP-43 in the cytoplasm by blue light illumination, in a zebrafish expressing the optoTDP-43 in almost all motor neurons.

FIG. 10A shows observation results showing swim bladder inflation failure by blue light illumination, in a zebrafish expressing optoTDP-43^{A315T} in almost all motor neurons.

FIG. 10B is a graph showing a formation proportion of the swim bladder inflation failure by blue light illumination, in a zebrafish expressing optoTDP-43^{A315T} in almost all motor neurons.

### DESCRIPTION OF EMBODIMENTS

<<Fusion Protein>>

A fusion protein of the present invention includes any one of proteins of (A) to (C) below; and a tag protein that dimerizes or multimerizes in response to a stimulus.

(A) A protein that consists of an amino acid sequence represented by SEQ ID NO: 1

(B) A protein that consists of an amino acid sequence having 70% or more identity with the amino acid sequence represented by SEQ ID NO: 1 and has an aggregate-forming ability

(C) A protein that consists of an amino acid sequence obtained by performing deletion, substitution, insertion, or addition of one or several amino acids with respect to the amino acid sequence represented by SEQ ID NO: 1 and has the aggregate-forming ability

An amino acid sequence represented by SEQ ID NO: 1 is an amino acid sequence of a zebrafish Tardbp protein. The zebrafish Tardbp protein is a homolog of a human TDP-43 protein. An amino acid sequence of the human TDP-43 protein is represented by SEQ ID NO: 2.

In a zebrafish, there are two homologs of Tardbp and Tardbpl, and an amino acid sequence of the zebrafish Tardbpl protein is represented by SEQ ID NO: 3.

An identity of an amino acid level between the zebrafish Tardbp protein and the human TDP-43 protein is 73%, and an identity of the amino acid level between the zebrafish Tardbp protein and the zebrafish Tardbpl protein is 78%. Any one of proteins of (A) to (C) also includes a protein consisting of the amino acid sequences represented by SEQ ID NOs: 1 to 3.

In (B), the identity is more preferably 75% or more, still more preferably 80% or more, particularly preferably 85% or more, and most preferably 90% or more.

In (C), the number of amino acids deleted, substituted, inserted, or added is preferably 1 to 120, more preferably 1 to 60, still more preferably 1 to 20, particularly preferably 1 to 10, and most preferably 1 to 5.

In the present invention, the “aggregate-forming ability” refers to an ability to form an atypical massive cellular component that can be detected using an optical microscope such as a confocal laser scanning microscope or a cellular component detected as an insoluble fraction in a cell extract, which is expected to be formed through an intermediate step of forming a multimer of a dimer or higher.

The tag protein that dimerizes or multimerizes in response to a stimulus refers to a protein containing a functional domain of a protein that forms a dimer or a multimer under light illumination or in the presence of a compound.

A set of the tag proteins that form a heterodimer under light illumination includes a set of PhyB and PIF, a set of FKF1 and GI, a set of CRY2 and CIB1, a set of UVR8 and COP1, a set of VVD and WC1, a set of PhyB and CRY1, and a set of RpBphP1 and RpPpsR2.

Examples of the tag proteins that form a homodimer under light illumination include UVR8, EL222, bPac, RsLOV, PYP, H-NOXA, YtvA, NifL, FixL, RpBphPI, and CRY2.

Examples of a set of tag proteins that form a heterodimer in the presence of a compound include a set of FKBP (FK506-binding protein) and FRB (FKBP12-rapamycin associated protein 1 fragment) in the presence of rapamycin, a system using gibberellin (compound) and a binding protein (GAI/GID1) thereto, a system using fusicoccin (compound) and a binding protein (CT52M1/T14-3-3cΔC-M2) thereto, a system using abscisic acid (compound) and a binding protein (PYL/ABI) thereto, and a system using rCD1/FK506 (compound) and a binding protein (FKBP/SNAP) thereto.

Among the tag proteins, for example, a mutant fragment of a cryptochrome CRY2 which has an activity of absorbing blue light and self-associating and is derived from Arabidopsis thaliana can be mentioned.

Examples of the mutant fragment of the cryptochrome CRY2 include a protein consisting of any one of the following amino acid sequences of (D) to (F).

(D) A protein that consists of an amino acid sequence represented by SEQ ID NO: 4

(E) A protein that consists of an amino acid sequence having 80% or more identity with the amino acid sequence represented by SEQ ID NO: 4 and has the activity of absorbing blue light and self-associating

(F) A protein that consists of an amino acid sequence obtained by performing deletion, substitution, insertion, or addition of one or several amino acids with respect to the amino acid sequence represented by SEQ ID NO: 4 and has the activity of absorbing blue light and self-associating

In (E), the identity is more preferably 85% or more, still more preferably 90% or more, and particularly preferably 95% or more.

In (F), the number of amino acids deleted, substituted, inserted, or added is preferably 1 to 100, more preferably 1 to 60, still more preferably 1 to 20, particularly preferably 1 to 10, and most preferably 1 to 5.

The fusion protein of the present invention preferably further includes a labeled protein. The labeled protein is not particularly limited as long as the expression of the fusion protein in a cell can be confirmed. Examples of the labeled protein include an epitope sequence of an antibody and a fluorescent protein, and the fluorescent protein is preferable from a viewpoint of observing a cell alive.

Examples of the fluorescent protein include a green fluorescent protein (GFP), a red fluorescent protein (RFP), a cyanide fluorescent protein (CFP), and a yellow fluorescent protein (YFP).

Examples of the fusion protein including the labeled protein include a protein consisting of an amino acid sequence represented by SEQ ID NO: 5.

<<Gene Encoding Fusion Protein>>

A gene of the present invention is a gene encoding the fusion protein of the present invention.

Examples of the gene include a gene encoding a protein that consists of any one of nucleotide sequences of the following (G) to (K) and has the aggregate-forming ability and a gene encoding the tag protein that dimerizes or multimerizes in response to a stimulus.

(G) A nucleotide sequence represented by SEQ ID NO: 6

(H) A nucleotide sequence obtained by performing deletion, substitution, insertion, or addition of one or several nucleotides with respect to the nucleotide sequence represented by SEQ ID NO: 6

(I) A nucleotide sequence in which an identity in the nucleotide sequence represented by SEQ ID NO: 6 is 70% or more, preferably 75% or more, more preferably 80% or more, further preferably 85% or more, and particularly preferably 90% or more

(J) A nucleotide sequence capable of hybridizing with a gene consisting of a nucleotide sequence complementary to the gene consisting of the nucleotide sequence represented by SEQ ID NO: 6, under a stringent condition

(K) Degenerate isomers of the nucleotide sequences of (G) to (J)

In (H), the number of nucleotides that may be deleted, substituted, inserted, or added is preferably 1 to 370, more preferably 1 to 180, still more preferably 1 to 60, particularly preferably 1 to 130, and most preferably 1 to 15.

Examples of a case “under a stringent condition” in (J) can include a condition in which hybridization is performed by performing incubation at 55° C. to 70° C. for several hours to overnight in a hybridization buffer formed of 5×SSC (composition of 20×SSC: 3M sodium chloride and 0.3M citric acid solution, and pH of 7.0), 0.1% by weight N-lauroyl sarcosine, 0.02% by weight of SDS, 2% by weight of blocking reagent for nucleic acid hybridization, and 50% formamide. A washing buffer to be used during washing after the incubation is preferably a 1×SSC solution containing 0.1% by weight of SDS, and more preferably a 0.1×SSC solution containing 0.1% by weight of SDS.

Amino acids other than methionine and tryptophan have multiple codons corresponding to one amino acid. This correspondence is called a degeneracy of a genetic code. In (K), the degenerate isomers of the nucleotide sequences mean other nucleotide sequences corresponding to the amino acids encoded by a certain nucleotide sequence.

Examples of the gene encoding the tag protein that dimerizes or multimerizes in response to a stimulus include the gene encoding the protein. Examples of the gene include a mutant fragment of a gene encoding cryptochrome which has an activity of absorbing blue light and self-associating and is derived from Arabidopsis thaliana.

Examples of the mutant fragment of the gene encoding the cryptochrome include a gene encoding a protein that consists of any one of nucleotide sequences of the following (L) to (P) and has the activity of absorbing blue light and self-associating.

(L) A nucleotide sequence represented by SEQ ID NO: 7

(M) A nucleotide sequence obtained by performing deletion, substitution, insertion, or addition of one to several nucleotides with respect to the nucleotide sequence represented by SEQ ID NO: 7

(N) A nucleotide sequence in which an identity in the nucleotide sequence represented by SEQ ID NO: 7 is 80% or more, preferably 85% or more, more preferably 90% or more, and particularly preferably 95% or more

(O) A nucleotide sequence capable of hybridizing with a gene consisting of a nucleotide sequence complementary to the gene consisting of the nucleotide sequence represented by SEQ ID NO: 7, under a stringent condition

(P) Degenerate isomers of the nucleotide sequences of (L) to (O)

Furthermore, examples of the gene encoding the fusion protein including the labeled protein include a gene consisting of a nucleotide sequence represented by SEQ ID NO: 8.

<<Vector>>

A vector of the present invention includes the gene of the present invention.

The vector is not particularly limited, and known vectors of the related art such as a plasmid vector and a viral vector can be mentioned. Examples of the plasmid vector include a vector having a promoter for expression in animal cells, such as a CAG promoter, an EF1α promoter, an SRα promoter, an SV40 promoter, an LTR promoter, a CMV (cytomegalovirus) promoter, and an HSV-tk promoter.

Examples of the viral vector include a retroviral vector, an adenoviral vector, an adeno-related viral vector, a vaccinia viral vector, a lentiviral vector, a herpesviral vector, an alpha viral vector, an EB viral vector, a papillomaviral vector, and a foamy viral vector.

<Cell and Non-Human Animal>>

A cell of the present invention includes: the fusion protein of the present invention, the gene of the present invention or a transcript thereof, or the vector of the present invention.

Examples of an organism from which the cell of the present invention is derived include mammals such as humans, monkeys, dogs, cats, rabbits, pigs, cows, mice, rats, and hamsters. Furthermore, examples thereof include general vertebrates, and include fish, amphibians, birds, and reptiles. Also, examples of thereof include invertebrates such as Drosophila, or yeasts.

Examples of a host used for the cell of the present invention include nervous system cells such as a glial cell, a nerve cell, an oligodendrocyte, a microglia, and an astrocyte.

In addition, a nervous system cell differentiated from a stem cell may be used as the host. The stem cell is a cell that has an ability to replicate oneself and an ability to differentiate into other cells of multiple strains. Examples of the stem cell include an embryonic stem cell (ES cell), an embryonic tumor cell, an embryonic germ stem cell, an induced pluripotent stein cell (iPS cell), a neural stem cell, a hematopoietic stem cell, a mesenchymal stem cell, a liver stem cell, a pancreatic stem cell, a muscle stem cell, a reproductive stem cell, an intestinal stein cell, a cancer stem cell, and a hair follicle stem cell.

As a method for introducing the fusion protein of the present invention, the gene of the present invention or a transcript thereof, or the vector of the present invention into the host, a suitable method for a live cell to be used can be used, and examples thereof include an electroporation method, a heat shock method, a calcium phosphate method, a lipofection method, a DEAE dextran method, a microinjection method, a particle gun method, a method using virus, and a method using a commercially available transfection reagent such as FuGENE (registered trademark) 6 Transfection Reagent (manufactured by Roche), Lipofectamine 2000 Reagent (manufactured by Invitrogen), Lipolectamine LTX Reagent (manufactured by Invitrogen), and Lipofectamine 3000 Reagent (manufactured by Invitrogen).

In addition, a non-human animal of the present invention includes: the fusion protein of the present invention, the gene of the present invention or a transcript thereof, or the vector of the present invention.

As the non-human animal, mammals or fish are preferable. Examples of the non-human mammals include mice, rats, guinea pigs, hamsters, rabbits, goats, pigs, dogs, and cats, and rodents such as mice and rats are preferable. As the fish, a zebrafish is preferable.

As will be described later in Examples, it has been confirmed that the fusion protein of the present invention can complement a function of wild-type TDP-43. Therefore, from a viewpoint closer to physiological conditions, in the cell or the non-human animal of the present invention, it is preferable that expressions of a tardbp gene or a homolog thereof, and a tardbpl gene or a homolog thereof are suppressed or lost, or functions of a Tardbp protein or a homolog thereof and a Tardbpl protein or a homolog thereof are suppressed or lost.

The expression that the “functions of a Tardbp protein and a Tardbpl protein are suppressed” refers to a state that original functions of the Tardbp protein and the Tardbpl protein are partially lost.

The expression that the “functions of a Tardbp protein and a Tardbpl protein are lost” refers to a state that original functions of the Tardbp protein and the Tardbpl protein are completely lost.

The suppression or loss of the functions of the Tardbp protein and the Tardbpl protein can also occur due to suppression or loss of the expressions of the tardbp gene and the tardbpl gene.

The expression that the “expressions of the tardbp gene and the tardbpl gene are suppressed” refers to a state that the amount of the tardbp gene product and tardbpl gene product in the cell or the animal of the present invention is suppressed as compared with a wild-type cell or animal as control.

The suppression of the expression of the tardbp gene and the tardbpl gene can be caused by introducing a nucleic acid sequence that causes expression of RNAi-inducible nucleic acid, antisense nucleic acid, aptamer, or ribozyme for the tardbp gene and the tardbpl gene into a cell or an animal and knocking down the gene.

The expression that the “expressions of the tardbp gene and the tardbpl gene are lost” refers to a state that the tardbp gene product and the tardbpl gene product are lost in the cell or the animal.

The loss of functions of the Tardbp protein and the Tardbpl protein which are the gene products can be caused, for example, by introducing a mutation into the tarbdp gene and the tardbpl gene and disrupting the tardbp gene and the tardbpl gene.

The mutations can be caused by deletion, substitution, insertion of a predetermined sequence, and the like of the tardbp gene and the tardbpl gene, or a part or all of an expression regulatory region of the genes. The introduction of the mutation thereof can be performed by using, for example, a treatment with a mutagenic substance, ultraviolet illumination, gene targeting by a homologous recombination technique or the like, gene knockout, conditional knockout by a Cre-loxP system or the like. In addition, a genome editing technology may be used for the gene targeting and the gene knockout.

The cell or the non-human animal of the present invention is useful as a TDP-43 proteinopathy model, because the protein forms a dimer or a multimer in response to a stimulus and forms an aggregate in a cytoplasm. Examples of the TDP-43 proteinopathy model include an amyotrophic lateral sclerosis, frontotemporal lobar degeneration model, a Parkinson's disease model, and an Alzheimer's disease model, and the amyotrophic lateral sclerosis model or a frontotemporal lobar degeneration model are preferable.

<<Screening Method>>

A screening method of the present invention is a method including: bringing or administrating the cell of the present invention or the non-human animal of the present invention into contact with or with a test substance under the presence of a stimulus; and selecting a useful candidate substance for preventing or treating TDP-43 proteinopathies.

For example, a compound library is added to a medium, the cell or the zebrafish is illuminated with blue light, and an effect on cell proliferation or zebrafish growth is examined. More specifically, for example, the cell of the present invention is seeded in a well plate, or the zebrafish as the non-human animal of the present invention is introduced and incubated or bred for about 1 to 10 days in the presence of a compound library while promoting a dimer formation of the TDP-43 protein under the blue light illumination. Thereafter, for the cell, the number of living cells is analyzed by, for example, color development by reduction of a tetrazolium salt. As the tetrazolium salt, commercially available 3-[4,5-dimethylthiazol-2-yl]-2,5-diphenyltetrazolium bromide) (MTT) or the like can be used. For the zebrafish, the life and death or activity status of the zebrafish may be checked. A compound that maintains or enhances the cell proliferation although the dimer formation of the TDP-43 protein causes gradual cytotoxicity is a candidate for a prophylactic agent or a therapeutic agent for the TDP-43 proteinopathy.

<<Screening Kit>>

A prophylactic drug or therapeutic drug screening kit for TDP-43 proteinopathy of the present invention is a kit including the cell of the present invention or the non-human animal of the present invention.

The kit of the present invention may further include, in addition to the cell of the present invention or the non-human animal of the present invention, a multi-well plate or the like necessary for screening.

<<Screening Apparatus>>

A prophylactic drug or therapeutic drug screening apparatus for TDP-43 proteinopathy of the present invention is an apparatus including the cell of the present invention or the non-human animal of the present invention; a well plate containing any of the cell or the non-human animal; and a light illumination device.

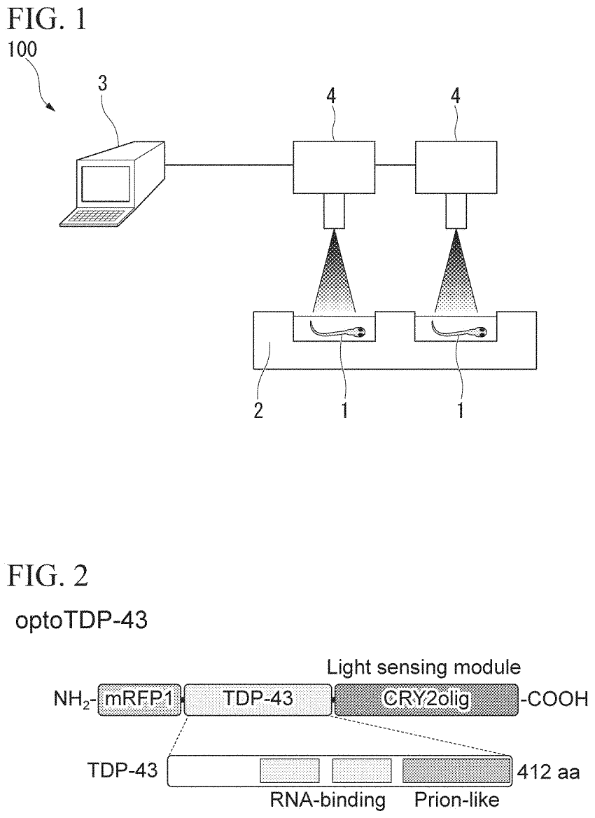

FIG. 1 is a schematic configuration diagram showing an example of the screening apparatus of the present embodiment. Each configuration of the screening apparatus of the present embodiment will be described in detail with reference to FIG. 1.

A screening apparatus 100 shown in FIG. 1 includes a TDP-43 proteinopathy model zebrafish 1 (hereinafter, zebrafish 1), a well plate 2, a control unit 3, and a light illumination device 4.

The zebrafish 1 expresses, in vivo, a fusion protein containing a mutant fragment of cryptochrome CRY2 which has an activity of absorbing blue light and self-associating and is derived from Arabidopsis thaliana, and a zebrafish Tardbp protein.

Each well of the well plate 2 contains a compound derived from a compound library, and the zebrafish 1 with an extension of 3 mm is swimming in water with which the well is filled.

The light illumination device 4 illuminates the well with blue light based on a command from the control unit 3.

The zebrafish is bred for about 1 to 10 days in the presence of the compound library while promoting the dimer formation of the Tardbp protein in the zebrafish 1, under the blue light illumination.

A compound present in the well where the zebrafish 1 whose activity is not affected is growing although the formation of the dimer or the multimer of the Tardbp protein causes gradual neurotoxicity is a candidate for a prophylactic agent or a therapeutic agent for TDP-43 proteinopathies.

Also, although not shown, the screening apparatus 100 may include a microscope for observing the zebrafish 1.

In addition, as another embodiment, a TDP-43 proteinopathy model cultured cell may be used instead of the zebrafish.

### EXAMPLES

Hereinafter, the present invention will be described with reference to Examples, but the present invention is not limited to the following examples.

[Construction of Fusion Protein Expression System]

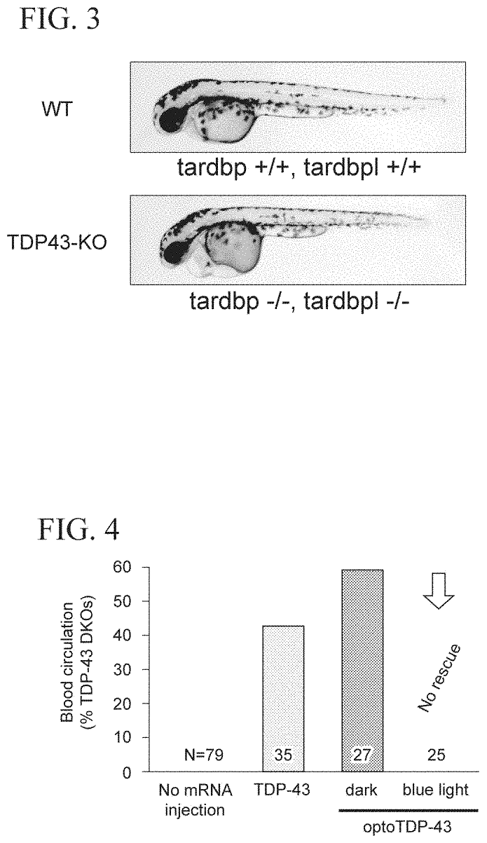

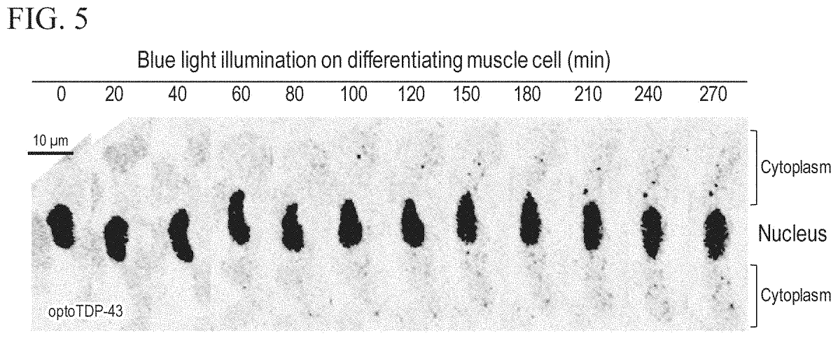

A gene construct expressing a fusion protein obtained by adding a mutant fragment (CRY2olig) of cryptochrome which has an activity of self-associating when absorbing blue light and is derived from Arabidopsis thaliana and a red fluorescent protein (mRFP1) to a TDP-43 (Tardbp) protein derived from the zebrafish was prepared (see FIG. 2). This fusion protein is referred to as optoTDP-43. An amino acid sequence of the optoTDP-43 is represented by SEQ ID NO: 5, and a nucleotide sequence of the gene encoding the optoTDP-43 is represented by SEQ ID NO: 8.

[Functional Test of Fusion Protein]

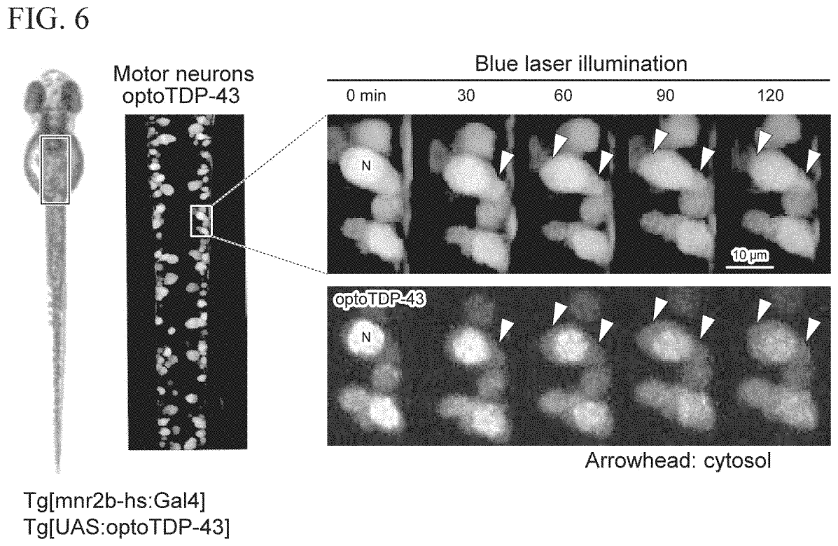

A zebrafish (tardbp −/−, tardbpl −/−) in which a tardbp gene and a tardbpl gene are knocked out was prepared, and it was confirmed that a phenotype in which blood circulation was impaired showed. An mRNA of a gene encoding the TDP-43 (Tardbp) protein derived from the zebrafish and an mRNA of a gene encoding the optoTDP-43 were injected into the double knockout zebrafish, respectively. In the zebrafish into which the mRNA of the gene encoding the TDP-43 (Tardbp) was introduced and the zebrafish into which the gene encoding the optoTDP-43 was introduced and which was bred in the dark, recovery of blood circulation was observed. However, in the zebrafish into which the mRNA of the gene encoding the optoTDP-43 was introduced and which was bred under blue light illumination, the recovery of blood circulation in the heart was not observed (see FIG. 4). From the fact, it was confirmed that the constructed optoTDP-43 has a function as the TDP-43, and that the function can be controlled by the presence or absence of blue light illumination.

[Observation for Transfer of TDP-43 Protein to Cytoplasm by Blue Light Illumination]

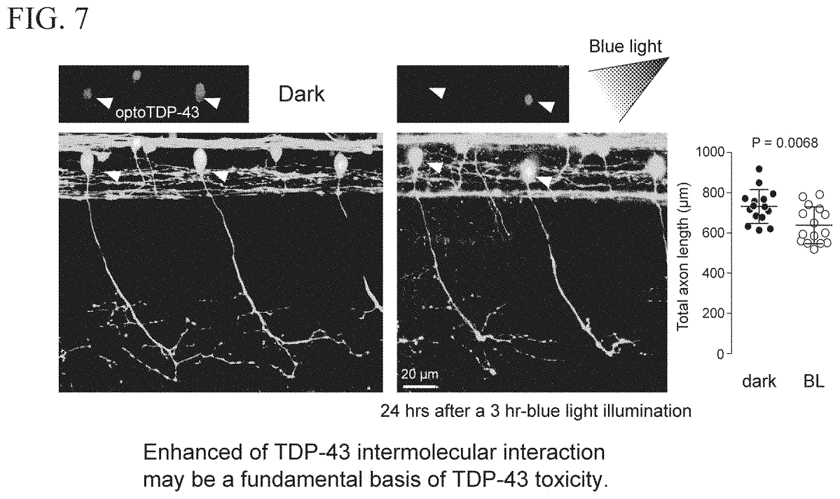

The zebrafish in which the mRNA of the gene encoding the optoTDP-43 was introduced into the double knockout zebrafish (tardbp −/−, tardbpl −/−) was continuously illuminated with blue light, and an intracellular localization of the optoTDP-43 in muscle cells was observed using the fluorescence of the mRFP1 as an index. As shown in FIG. 5, the transfer of the optoTDP-43 to the cytoplasm was confirmed in a time-dependent manner. Furthermore, since the red fluorescence showed a dot shape in the cytoplasm, it was confirmed that the optoTDP-43 formed an aggregate.

[Observation of Transfer of TDP-43 Protein to Cytoplasm by Blue Light Illumination, in Zebrafish in which Fusion Protein is Expressed Specifically for Spinal Motor Neurons and Observation of Axon Elongation]

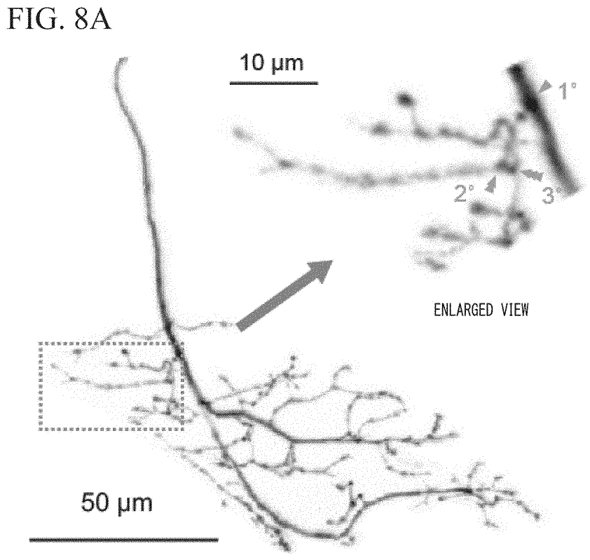

A zebrafish in which the optoTDP-43 was expressed only in spinal motor neurons was constructed by introducing Bacterial artificial chromosome (BAC), which incorporates optoTDP-43 under the promoter of mnr2b gene, into the wild type (see Protocadherin-Mediated Cell Repulsion Controls the Central Topography and Efferent Projections of the Abducens Nucleus. Asakawa K, Kawakami K. Cell Reports. 2018 24: p 1562-1572). The zebrafish was continuously irradiated with blue light, and the transfer of the optoTDP-43 in the spinal motor neurons was observed using the fluorescence of mRFP1 as an index. As shown in FIG. 6, it was confirmed that the amount of the optoTDP-43 localized in the nucleus decreased and that the optoTDP-43 was transferred to the cytoplasm in a time-dependent manner.

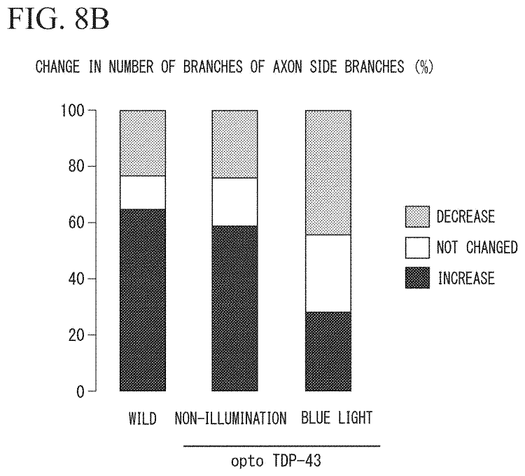

A zebrafish, which promoted the transfer of the optoTDP-43 to the cytoplasm by illumination with blue light, was bred again under a dark condition, thereafter, an axon elongation was observed. As a result, the axon elongation was inhibited (See FIG. 7). Therefore, it was confirmed that transient promotion of the transfer of the optoTDP-43 to the cytoplasm causes toxicity to the spinal motor neurons.

[Observation of Neuromuscular Synapses Once Formed by Blue Light Illumination, in Zebrafish in which Fusion Protein was Expressed Specifically for Spinal Motor Neurons]

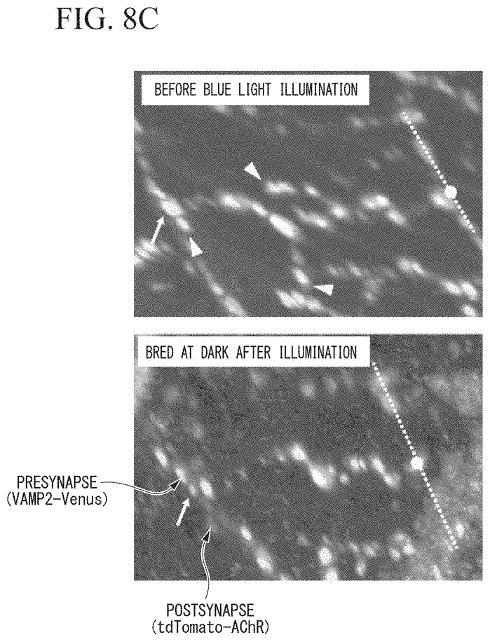

Furthermore, using a zebrafish in which the optoTDP-43 was expressed only in spinal motor neurons, fry 56 hours after fertilization in which side branches of the motor neurons were formed were illuminated with blue light for 3 hours, and then bred in the dark for 13 hours and axon side branches and changes in neuromuscular synapses were observed. As shown in FIG. 8A, the number of axon side branches was measured. The results thereof are shown in FIG. 8B.

As shown in FIG. 8B, it was confirmed that the motor neurons that experienced photic stimulation of the optoTDP-43 increased the rate of decrease in the number of axon terminals. In addition, the localization of presynapses and postsynapses was confirmed, as a result, it was confirmed that collapse of neuromuscular synapses was also promoted (see FIG. 8C).

[Observation of Aggregation of optoTDP-43 and TDP-43 by Blue Light Illumination in Zebrafish Expressing Fusion Protein Specifically for Motor Neurons]

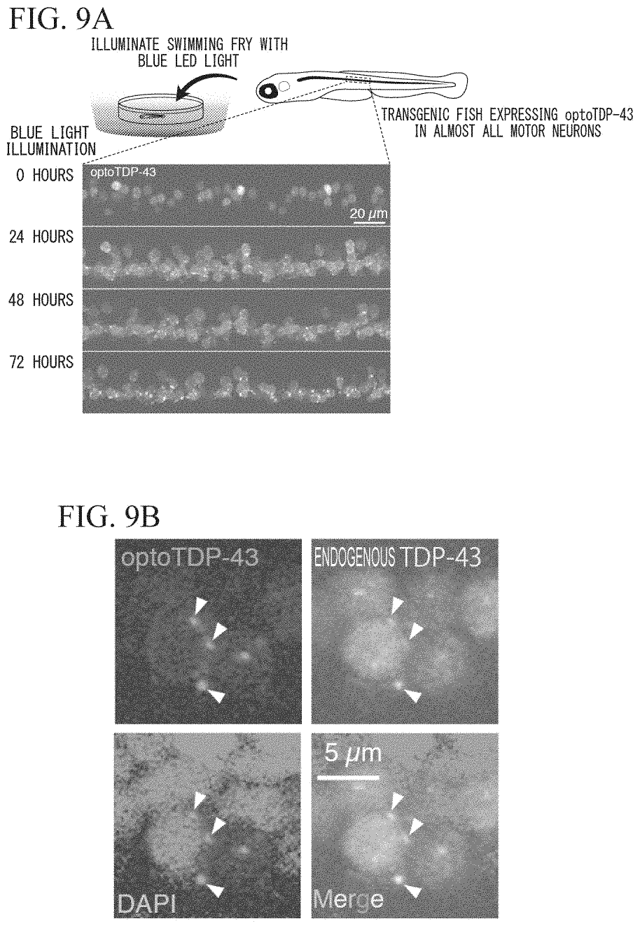

Using the BAC strain incorporating the optoTDP-43, a zebrafish strain expressing the optoTDP-43 in almost all motor neurons was constructed, and the constructed zebrafish strain was bred on a blue LED light panel, and aggregate of the optoTDP-43 and the TDP-43 agglomeration was observed. As shown in FIG. 9A, induction of aggregation of the optoTDP-43 in the cytoplasm was confirmed by long-term photic stimulation. As shown in FIG. 9B, at this time, the endogenous TDP-43 was also involved in the aggregation, and the propagation of the aggregation was confirmed.

[Observation of Movement Disorder Induction by Blue Light Illumination in Zebrafish Expressing Mutant (A315T) Fusion Protein Specifically for Motor Neurons]

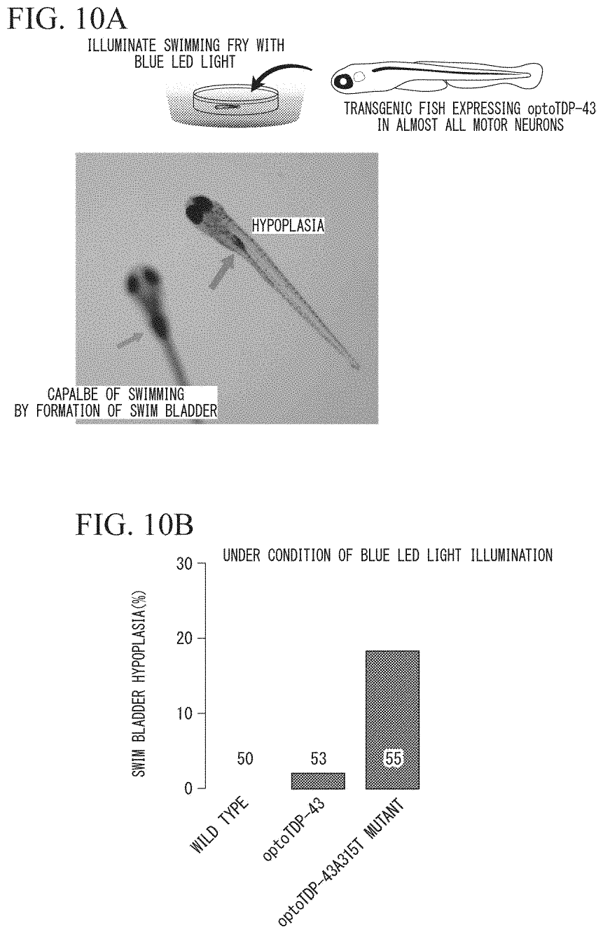

A strain expressing optoTDP-43^{A315T}, which has the introduced A315T mutation found in familial ALS was established in almost all motor neurons, and changes in motility due to the blue light illumination were observed. As shown in FIG. 10A, the introduction of the A315T mutation confirmed hypoplasia of the swim bladder. As shown in FIG. 10B, an increase in hypoplasia of the swim bladder was confirmed in the optoTDP-43^{A315T} as compared with the optoTDP-43. Since the formation of the swim bladder requires the development of normal motor ability, it can be said that light illumination induces movement disorders.

As above, it was confirmed that the optoTDP-43 can be widely used from cultured cells to non-human animals, and can be applied to the reproduction of temporally and spatially controlled pathological conditions and the development of drugs that alleviate the toxicity of TDP-43.

### INDUSTRIAL APPLICABILITY

According to the present invention, it is possible to provide a TDP-43 proteinopathy model that reflects a pathological condition of the TDP-43 proteinopathy.

1. A fusion protein comprising:
any one of proteins of (A) to (C) below; and
a tag protein that dimerizes or multimerizes in response to a stimulus,
(A) a protein that consists of an amino acid sequence represented by SEQ ID NO: 1,
(B) a protein that consists of an amino acid sequence having 70% or more identity with the amino acid sequence represented by SEQ ID NO: 1 and has an aggregate-forming ability, and
(C) a protein that consists of an amino acid sequence obtained by performing deletion, substitution, insertion, or addition of one or several amino acids with respect to the amino acid sequence represented by SEQ ID NO: 1 and has the aggregate-forming ability.

2. The fusion protein according to claim 1, further comprising a labeled protein.

3. A gene encoding the fusion protein according to claim 1.

4. A vector comprising the gene according to claim 3.

5. A cell comprising: the fusion protein according to claim 1.

6. The cell according to claim 5,
wherein expressions of a tardbp gene or a homolog thereof, and a tardbpl gene or a homolog thereof are suppressed or lost, or functions of a Tardbp protein or a homolog thereof and a Tardbpl protein or a homolog thereof are suppressed or lost.

7. A non-human animal comprising: the fusion protein according to claim 1.

8. The non-human animal according to claim 7,
wherein expressions of a tardbp gene or a homolog thereof, and a tardbpl gene or a homolog thereof are suppressed or lost, or functions of a Tardbp protein or a homolog thereof and a Tardbpl protein or a homolog thereof are suppressed or lost.

9. A TDP-43 proteinopathy model comprising the cell according to claim 5,
wherein the protein forms a dimer or a multimer in response to a stimulus.

10. The TDP-43 proteinopathy model according to claim 9, which is a model of amyotrophic lateral sclerosis or frontotemporal lobar degeneration.

11. A screening method comprising:
bringing or administrating the cell according to claim 5 into contact with or with a test substance under a presence of a stimulus; and
selecting a useful candidate substance for preventing or treating TDP-43 proteinopathies.

12. A prophylactic drug or therapeutic drug screening kit for TDP-43 proteinopathy, the kit comprising:
the cell according to claim 5.

13. A prophylactic drug or therapeutic drug screening apparatus for TDP-43 proteinopathy, the apparatus comprising:
the cell according to claim 5;
a well plate containing any of the cell or the non-human animal; and
a light illumination device.
